(19) United States
(12) Patent Application Publication
(10) Pub. No.: US 2004/0215196 A1
(43) Pub. Date: Oct. 28, 2004

(54) Cervical fusion screw guide

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 10/421,690

(22) Filed: Apr. 24, 2003

(51) Int. Cl. A61B 17/56

(52) U.S. Cl. 606/72

(57) ABSTRACT

A cervical fusion screw guide includes a tube through which a drill or a bone screw can be passed, and a needle-like probe which is offset from the axis of the tube. The probe may extend parallel to or at an angle oblique to the tube axis. An assortment of the guides, having different offset distances and differing angles, are provided. Alternatively, a single adjustable probe, having a variable probe angle and/or a variable probe offset, may be used.

### BACKGROUND OF THE INVENTION

This invention relates to a screw guide which well improve the accuracy of cervical plate placement in the performance of anterior cervical fusion surgery.

Of all animals possessing a backbone, human beings are the only creatures who remain upright for significant periods of time. From an evolutionary standpoint, this erect posture has conferred a number of strategic benefits, not the least of which is freeing the upper limbs for purposes other than locomotion. From an anthropologic standpoint, it is also evident that this unique evolutionary adaptation is a relatively recent change and as such has not benefitted from natural selection as much as have backbones held in the horizontal attitude. As a result, the stresses acting upon the human backbone (or “vertebral column”), are unique in many senses, and result in a variety of problems or disease states that are peculiar to the human species.

The human vertebral column is essentially a tower of bones held upright by fibrous bands called ligaments and contractile elements called muscles. There are seven bones in the neck or cervical region, twelve in the chest or thoracic region, and five in the low back or lumbar region. There are also five bones in the pelvis or sacral region which are normally fused together and form the back part of the pelvis. This column of bones is critical for protecting the delicate spinal cord and nerves, and for providing structural support for the entire body.

Between the vertebral bones themselves exist soft tissue structures—discs—composed of fibrous tissue and cartilage which are compressible and act as shock absorbers for sudden downward forces acting on the upright column. More importantly, the discs allow the bones to move independently of each other to permit functional mobility of the column of spinal vertebrae. Unfortunately, the repetitive forces act on these intervertebral discs during repetitive day-to-day activities of bending, lifting and twisting cause them to break down or degenerate over time.

Presumably because of humans' upright posture, their intervertebral discs have a high propensity to degenerate. Overt trauma, or covert trauma occurring in the course of repetitive activities disproportionately affect more highly mobile areas of the spine. Disruption of a disc's internal architecture leads to bulging, herniation or protrusion of pieces of the disc and eventual disc space collapse. Resulting mechanical and even chemical irritation of surrounding neural elements (spinal cord and nerves) cause pain, attended by varying degrees of disability. In addition, loss of disc space height relaxes tension on the longitudinal spinal ligaments, thereby contributing to varying degrees of spinal instability such as spinal curvature or lithesis.

The time-honored method of addressing the issues of neural irritation and instability resulting from severe disc damage have largely focused on removal of the damaged disc and fusing the adjacent vertebral elements together. Removal of the disc relieves mechanical and chemical irritation of neural elements, while osseous union (bone knitting) solves the problem of instability.

In the cervical spine, the most common type of fusion utilizes either bone dowels (Cloward Technique) or bone blocks (Smith-Robinson Technique). The procedures have been used now for over four decades. One of the main causes of failure of these fusion techniques is the failure to fuse, or non-union, at the site where the bone is grafted between the vertebral bodies. In an attempt to overcome this problem, various plate-type mechanisms have been used both to provide immediate stability, and to reduce or eliminate movement at the site of the fusion to allow successful bone knitting, much as a cast on a fractured limb provides support until healing can occur.

It is recognized that for bone knitting to occur, the interfaces of bone required to knit or heal must be held in close apposition, and motion between the knitting or fusion interfaces must be restricted sufficiently for a certain minimal time period to permit stable bone growth to occur.

One of the problems with the use of a cervical plate to aid in spinal fusion is plate placement. Because the plate is affixed to bone with a variety of bone screws, it is critical that the screws be accurately placed so that maximum bone purchase can be achieved and so that screws are not inadvertently placed into the disc space where the soft tissues confined therein will not provide adequate long term support for a screw. Most present plating systems require the plate to be placed over the spine before screw placement. Once the plate is placed over the spine and the constructs to be immobilized, visual assessment of the bony element in relation to the disc is obscured. This situation can lead to less than ideal screw placement as considerable judgment must then be employed on the part of the operating surgeon, who must now choose an acceptable trajectory for the screw while lacking some of the visual feedback that would be provided if the plate were not there.

### SUMMARY OF THE INVENTION

It is the object of this invention to provide a cervical fusion screw guide which aids in accurate bone screw placement and thereby improves the success of anterior cervical plating procedures.

To achieve this objective, a cervical screw guide is employed. This screw guide has a cylindrical tubular structure with an offset pin or probe which is designed to be pressed or placed into the soft tissue of the disc above or below the area of the spine to be fused.

The central axis of the tube is a fixed distance from the central axis of the pin or probe which is pressed into the disc. These axes are generally parallel, although not precisely so: changing the relative alignment of these axes allows one to control the angle of screw placement relative to the plane of the vertebral end plate.

The distance between the central axis of the cylinder or guide tube and the offset pin ultimately determines the distance at which the central screw is placed from the disc space, or, more accurately, from the end plate of the disc space.

The guide tube is utilized to direct a drill and/or screw to the exact desired distance below the end plane, thereby preventing inadvertent screw placement into the disc space itself. In this manner, accurate screw placement relative to the disc space is assured.

### BRIEF DESCRIPTION OF THE DRAWINGS

In the accompanying drawings,

FIG. 1 is a of a cervical fusion screw guide embodying the invention;

FIG. 2 some of the disc and their adjacent end plate have been removed in preparation for spinal fusion;

FIG. 3 illustrates the bone grafts to be in their appropriate position;

FIGS. 4a and 4b show alternative designs of the probe portion of the guide;

FIG. 5a-5f shows an assortment of tools having different geometry; and

FIG. 6 is an isometric view of an adjustable tool embodying the invention.

### DESCRIPTION OF THE PREFERRED EMBODIMENT

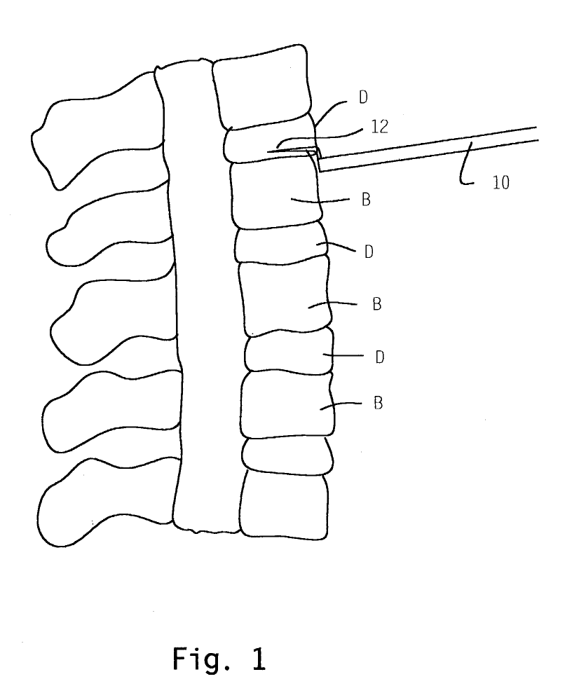

FIG. 1 shows a normal spine from the side; the spine includes a series of vertebral bodies “B” separated by discs “D”. A drill guide embodying the invention has been placed on the anterior side of the spine. It comprises a tube 10 and a sharply pointed probe 12 which is offset from and parallel to or slightly oblique to the axis of the tube. The amount of offset determines how far the axis of the tube will be from the end plate when the probe is inserted into one of the discs adjacent the end plate. The angle of the probe with respect to the centerline or axis of the tube determines the angularity of the tube.

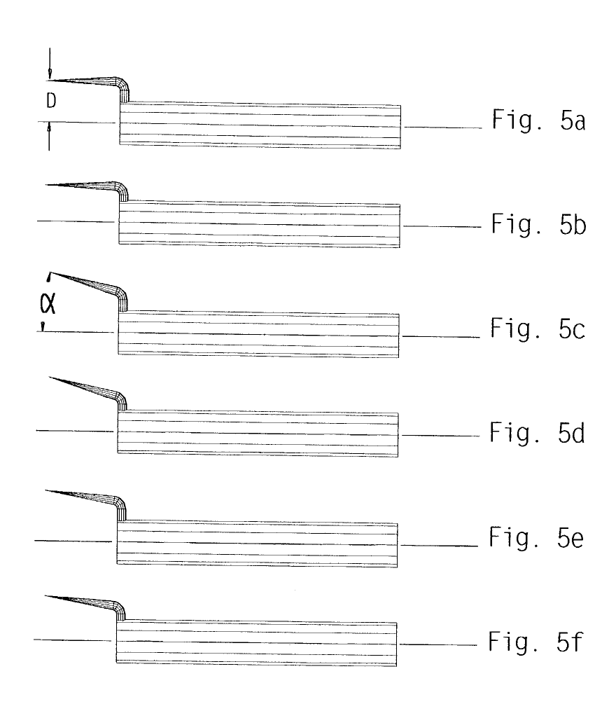

An assortment or set (FIG. 5) of such tools with different offset distances “X” between the tip of the probe and the axis of the tube, and with varying angles α between the probe axis and the tube axis, permit ranges of screw placement both in terms of the site of entry on the vertebral body and the angle of the screw placement within the body.

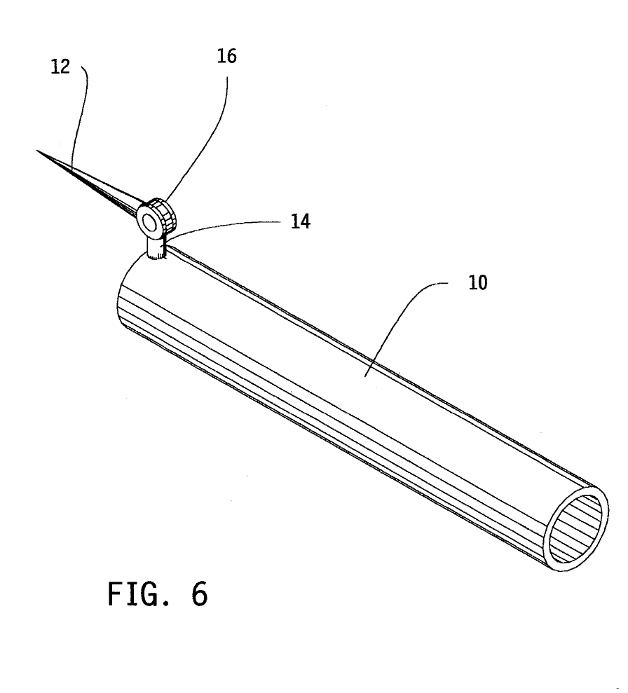

Alternatively, the offset distance and/or angle may be made adjustable in a single tool, as shown in FIG. 6, which shows a telescoping section 14 at the base of the probe, and a hinge 16 at the elbow of the probe. The location of the hinge may be at points other than that shown. This hinge must have substantial frictional resistance, or a locking mechanism (not shown) to prevent the probe from moving in use. The telescoping section may be threaded, or merely frictional, but must have adequate strength to resist the axial forces delivered along the probe.

To use of any of the drill guides described above, the tip of the probe is pushed into the body to be drilled, so that the tube contacts the body and is held securely in place. Then a drill bit is passed through the tube and is rotated to drill a hole of a predetermined depth in the vertebra. This procedure is performed above and below the fusion site.

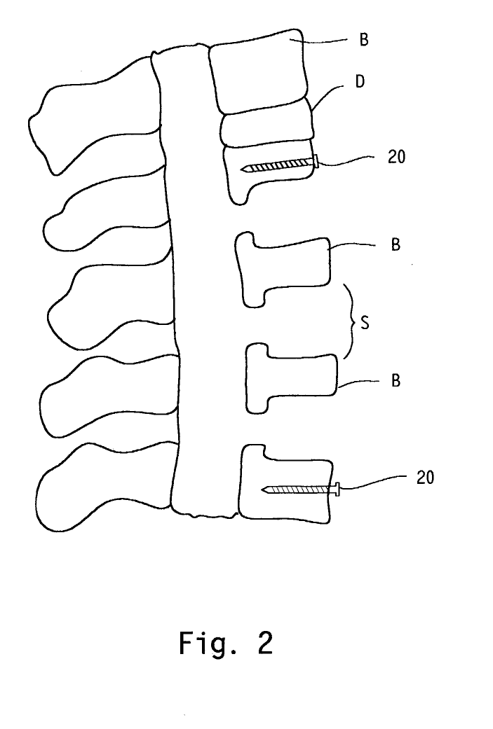

Next, screws 20 are threaded into the holes (FIG. 2). Because a precision guide was used while drilling the holes, the screws lie at precise distances from the disc spaces and are at proper predetermined angles to the end plate, so they do not extend into the enlarged spaces “S”, which are formed by removing the end plates from the adjacent bodies “B”.

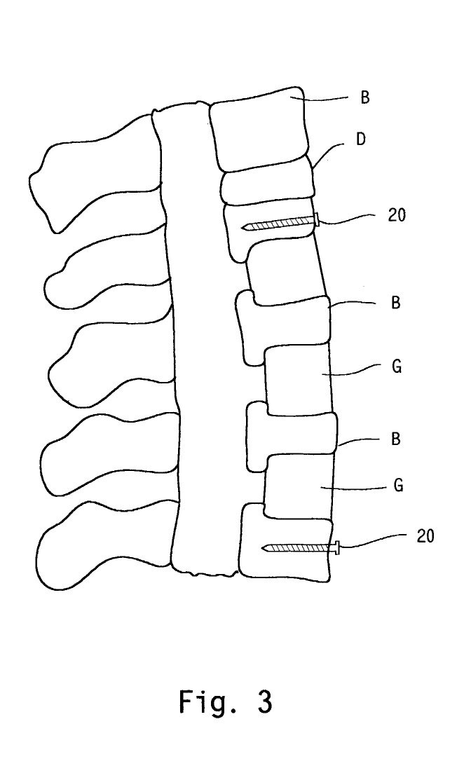

Finally, bone grafts “G” are inserted in the prepared spaces “S”, as shown in FIG. 3

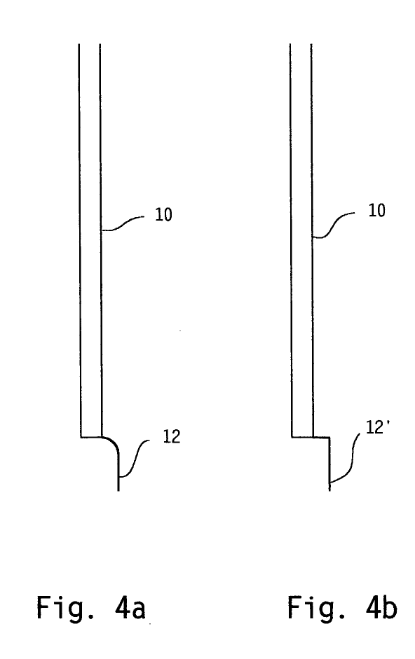

FIGS. 4a and 4b illustrates alternative designs of the probes 12, 12′ used to identify the disc and end plate.

Since the invention is subject to modifications and variations, it is intended that the foregoing description and the accompanying drawings shall be interpreted as only illustrative of the invention defined by the following claims.

I claim:

1. A drill guide for cervical fusion surgery, said guide comprising a tube having a longitudinal axis and a probe attached to the tube adjacent one end thereof, said probe having a sharply pointed tip, and being offset from the axis of the tube and extending parallel to or intersecting said axis.

2. The invention of claim 1, wherein the probe has sufficient strength to allow it to be pushed or pressed into a vertebral disc so that the drill guide tube abuts against the bone of the vertebral body at a location determined by the distance between the axis of the probe and guide tube and at an angle determined by that between the tube and the probe.

3. The invention of claim 1, wherein the probe has a lengthwise axis which intersects the axis of the tube at an acute angle.

4. The invention of claim 3, wherein the probe can be moved relative to the drill guide tube to adjust said angle.

5. The invention of claim 1, wherein the probe can be displaced relative to the drill guide tube to adjust the distance from the tip of the probe to the axis of the tube.

6. A set of drill guides for cervical fusion surgery, each guide comprising a tube extending along an axis and a probe attached to the tube, the probes of the respective drill guides being offset from the axes of their respective tubes by varying distances.

7. A set of drill guides for surgical fusion surgery, each guide comprising a tube extending along an axis and a probe attached to the tube, the probes of the respective drill guides extending from said tubes at varying angles.

8. A set of drill guides as recited in claim 7, wherein the probes of the respective drill guides are offset from the axes of their respective tubes by varying distances.
